(19) United States
(12) Patent Application Publication
(10) Pub. No.: US 2023/0414699 A1
(43) Pub. Date: Dec. 28, 2023

(54) TREATMENT OF DERMATOLOGICAL CONDITIONS

(71) Applicant: MC2 Therapeutics Ltd, Guildford (GB)

(72) Inventors: Torkil Menne, Guildford (GB); Johan Selmer, Guildford (GB); Jesper Lange, Guildford (GB); Jon Bondebjerg, Guildford (GB); Michelle Georgiou, Guildford (GB)

(73) Assignee: MC2 Therapeutics Ltd, Guilford (GB)

(21) Appl. No.: 18/248,137

(22) PCT Filed: Oct. 8, 2021
(86) PCT No.: PCT/EP2021/077931

§ 371 Date: Apr. 6, 2023

(30) Foreign Application Priority Data
Oct. 9, 2020 (EP) 20201040.1

(51) Int. Cl. A61K 38/05; A61K 38/06; A61K 31/198; A61K 31/4985; A61K 31/15; A61P 17/00

(52) U.S. Cl. CPC A61K 38/05; A61K 38/06; A61P 17/00; A61K 31/4985; A61K 31/15; A61K 31/198

(57) ABSTRACT

Provided is a pharmaceutical composition which comprises a nucleophilic compound capable of inhibiting carbamylation, for use in the treatment of a condition involving cutaneous or connective tissue damage. A method for treating a condition involving cutaneous or connective tissue damage in a patient, is also provided, said method comprising administering a said pharmaceutical composition to said patient.

### TECHNICAL FIELD

The present invention relates to a pharmaceutical composition comprising a nucleophilic compound capable of inhibiting carbamylation, for use in the treatment of a condition, in particular a dermatological condition, involving cutaneous or connective tissue damage. A method for treating a condition involving cutaneous or connective tissue damage in a patient is also provided, said method comprising administering a pharmaceutical composition comprising a nucleophilic compound capable of inhibiting carbamylation to said patient.

### TECHNICAL BACKGROUND

Lichen sclerosus (LS), also known as lichen sclerosus et atrophicus (LSA), is a chronic, inflammatory atrophic skin disease that affects mainly anogenital areas. LS can also be found on the inner thigh, buttocks, under breasts, neck, shoulders and armpits. Cases are found in both males and females, although females are more commonly affected. The etiology is unknown, but autoimmunity and changes in hormone levels (in particular estrogen levels) are thought to contribute to the condition.

Signs and symptoms of LS include pruritus, irritation, painful intercourse, dysuria, urethral and vaginal discharge, dyspareunia, urinary and fecal incontinence (Bunker C B et. al. 2013; Christmann-Schmid et. al. 2018). Painful skin fissuring, synechiae formation, clitoral phimosis, and thickening of skin may also occur in the effected region.

The skin in LS patients is characterized by an amorphous layer (hyalinization band) in the upper parts of the dermis with the appearance of a glassy hyaline layer. The hyalinization band consists of thick, distorted and fragmented collagen fibers and the absence of elastin fibers (Godoy et al 2015). The epidermis including the stratum corneum is atrophic. LS can lead to an increased risk of certain cancers (e.g. vulval cancer).

There are only a few treatments of LS available, including hormonal and corticosteroid treatments, and in many cases, these do not provide remission of symptoms. Most patients also manage their symptoms with topical moisturisers, but these do not treat the underlying condition. EP 3 498 276 describes the use of ectoine and ectoine derivatives for the treatment and/or prevention of vulvovaginal dermatologic conditions.

A need remains, therefore, for effective treatment of cutaneous or connective tissue conditions, such as e.g. lichen sclerosus, particularly vulval lichen sclerosus.

Carbamylation is a posttranslational modification of nucleophilic amino groups of proteins and amino acids by isocyanate. The reaction preferentially takes place on the α-amino groups of amino acids, peptides, or proteins, but also, at a 100 times lower speed due to their lower pKa, on ε-amino groups of lysine residue side chains. The reaction can also take place on thiol functional groups, and in some cases this may be reversible. The most common pathway leading to isocyanate formation is the spontaneous dissociation of urea into ammonia and isocyanate in aqueous solutions, which physiologically occurs in a ˜99:1 ratio in favor of urea. Isocyanate may also result from the transformation of thiocyanate by myeloperoxidase (MPO) in the presence of hydrogen peroxide (Wang et. al. 2007). Thiocyanate is introduced via diet, especially by fruits and vegetables as well as milk by-products, and by smoke. MPO is known to be an abundant enzyme contained in inflammatory cells such as polymorphonuclear neutrophils and monocytes/macrophages. Inflammation, smoking and uremia or reduced renal function have been reported to increase carbamylation (Wang et. al. 2007).

Protein carbamylation can lead to the loss of tolerance with formation of antibodies directed against carbamylated proteins (anti-CarP antibodies) in susceptible individuals (Shi et. al. 2014). Anti-CarP antibodies have been detected in serum of patients suffering from autoimmune, inflammatory diseases involving cutaneous or connective tissue manifestations, including but not limited to, Lupus erythematosus (Ziegelasch et.al. 2016) and systemic sclerosis, or scleroderma (Favoino et. al. 2018).

Collagen is the most abundant protein in the body and not only plays a structural role but also regulates cell growth, differentiation, and migration. Collagen is a major target of carbamylation due to its particularly slow turnover (Pietrement et al., 2013). Previous studies have reported that carbamylation has critical effects on the structure and function of collagen, including decreased thermal stability (Jaisson et al., 2006), disturbed fibril formation (Jaisson et al., 2006), altered susceptibility to collagenases (Jaisson et al., 2007), and decreased ability to activation of inflammatory cells (Jaisson et al., 2006, 2008). Recent results suggest that hydroxylysine carbamylation affects the mechanical properties of connective tissue by competitively inhibiting collagen cross-link formation in various tissue, including skin (Taga et. al. 2017).

### SUMMARY

It has been discovered that (in particular, dermatological) conditions involving cutaneous or connective tissue damage can be treated by inhibiting carbamylation. Specifically, it has been shown that a patient suffering from vulvar lichen sclerosus has achieved complete remission of symptoms, reduction of pain, and a dramatic improvement of skin changes after few weeks of treatment with a cream containing nucleophiles. There is a rationale that isocyanate, derived from urea, arising from associated urinary incontinence, and/or increased MPO-mediated transformation of thiocyanate due to inflammation, is a causal factor for lichen sclerosus due to destructive carbamylation of cutaneous and connective tissue (e.g. elastin, collagen). Without being bound by theory, the inventors believe that carbamylation affects the intramolecular bridges and the 3-dimensional structure of collagen, elastin and anchoring filaments, which results in the atrophy, fibrosis, fissures, and hyperkeratosis observed not only in LS patients, but also a range of other inflammatory diseases with cutaneous and connective tissue manifestations, including but not limited to, cutaneous Lupus erythematosus, localized scleroderma, lichen planus, Dupuytren's contracture, Carpal tunnel syndrome, morphea, acquired perforating dermatosis, and vulvovaginal atrophy.

Further, other recent studies have identified circulating autoantibodies against the Extracellular Matrix 1 protein (ECM1) in most patients with lichen sclerosus (Tran et. al. 2019). Within the epidermis, ECM1 has a role in the control of keratinocyte differentiation. ECM1 binds to the major heparan sulphate proteoglycan, perlecan. In this way, ECM1 may act as a ‘biological glue’ in the dermis, helping to regulate basement membrane and interstitial collagen fibril macro-assembly and growth factor binding. ECM1 may also have a role in other acquired skin disorders and physiological skin changes. It is conceivable that carbamylation may also lead to dysregulation of ECM1 and thereby contribute to cutaneous and connective tissue damage in lichen sclerosus and other conditions.

Another important protein for function of various tissues is filaggrin, which is particularly essential for epidermal homeostasis (Thyssen and Maibach 2014), Similarly, carbamylation of filaggrin may lead to loss of function, resulting in cutaneous and connective tissue damage.

A pharmaceutical composition is thus provided, said pharmaceutical composition comprising a nucleophilic compound capable of inhibiting carbamylation, for use in the treatment of a condition (in particular, a dermatological condition) involving cutaneous or connective tissue damage. A method for treating a dermatological condition, in particular a dermatological condition, involving cutaneous or connective tissue damage in a patient is also provided, said method comprising administering a pharmaceutical composition comprising a nucleophilic compound capable of inhibiting carbamylation to said patient.

Further details of the technology are provided in the following description text, examples and dependent claims.

### LEGENDS

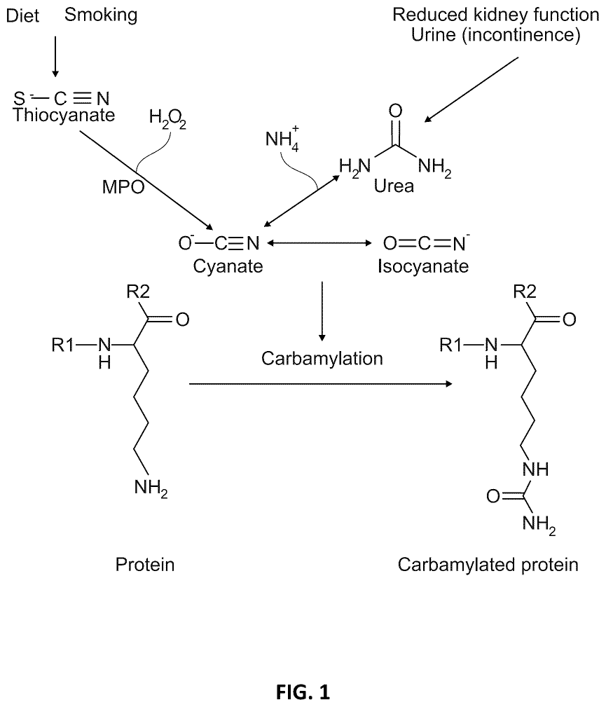

FIG. 1. Illustrates a mechanism by which cyanate or isocyanate, formed via different pathways, results in carbamylation of side chains of proteins which leads to altered, or loss of, function. The abbreviation MPO refers to myeloperoxidase.

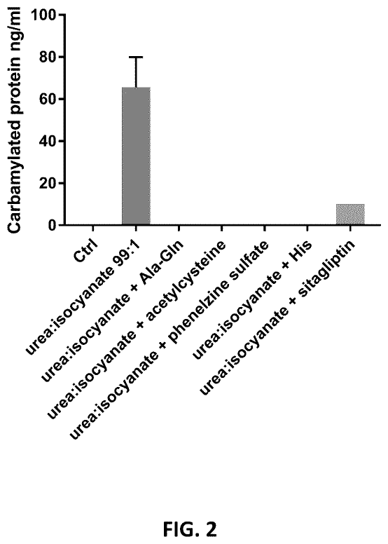

FIG. 2 shows results from in vitro protein carbamylation assay using reconstructed human skin.

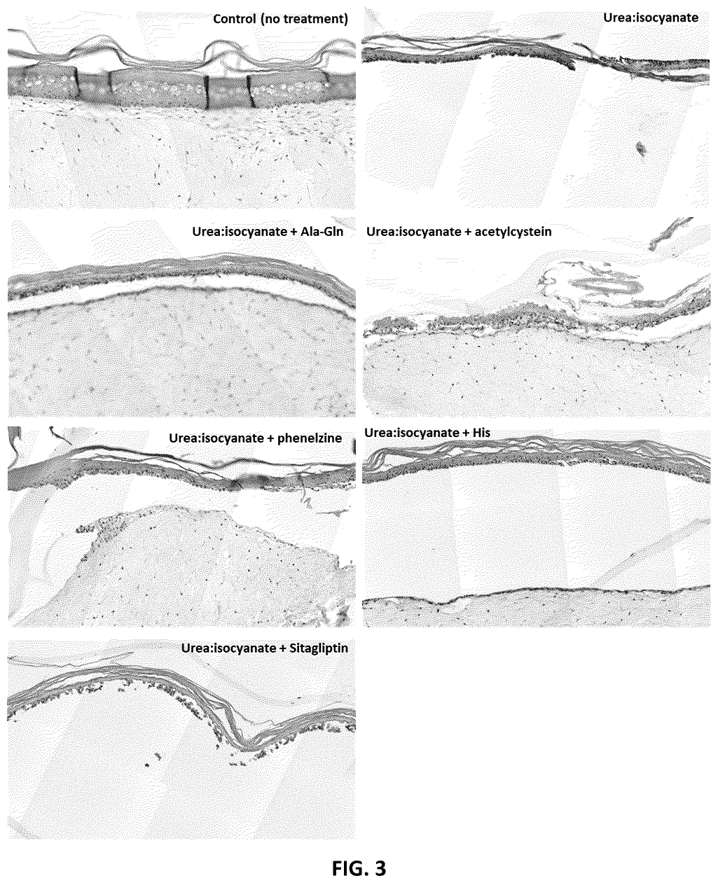

FIG. 3: Pictures of Hematoxylin and Eosin (H&E) staining of skin samples treated with various nucleophiles tested in FIG. 2.

### DETAILED DISCLOSURE

Pharmaceutical Composition

In a first aspect, a pharmaceutical composition is provided which comprises a nucleophilic compound capable of inhibiting carbamylation, for use in the treatment of a condition, in particular a dermatological condition, involving cutaneous or connective tissue damage. The nucleophilic compound is believed to inhibit carbamylation by scavenging the cyanate/isocyanate as illustrated in FIG. 1. Preferably, the composition is a topical composition.

The terms “nucleophile” and “nucleophilic compound” indicate an organic compound with a nucleophilic moiety which can donate an electron pair to another molecule or chemical moiety to form a chemical bond. All nucleophiles are Lewis bases. In the present technology, it is theorised that the nucleophilic moiety of the nucleophilic compound reacts with isocyanate present in the patient's skin, thus inhibiting protein carbamylation by isocyanate.

The nucleophilic properties of the nucleophilic compound can be determined by the degree of carbamylation of the model protein Bovine Serum Albumin (BSA) in the presence of said nucleophile

In one aspect, the nucleophilic compound results in a degree of BSA carbamylation (relative to the control in Hank's Balanced Salt Solution, HBSS) of less than 80%, preferably less than 70%, more preferably less than 60%, even more preferably less than 50% as measured by the in vitro protein carbamylation assay provided herein. According to this definition, compounds which do not provide the required degree of BSA carbamylation are not considered “nucleophilic compounds”.

As set out above, the nucleophilic compound is suitably an organic molecule comprising at least one nucleophilic moiety. More than one nucleophilic moiety, optionally more than one nucleophilic moiety of different types, may be present on the nucleophilic compound.

In one aspect, at least one nucleophilic moiety is selected from primary amine (—NH₂), secondary amine (—NHR₁—), guanidino (—NR₁C(NR₂)NR₃R₄), amidino (—C(NR₂)NR₃R₄), hydrazino (R₁—NR₂—NR₃R₄) or thiol (—SH).

To provide optimal inhibition of BSA carbamylation, at least one nucleophilic moiety is preferably in its unprotonated form, in said composition or said method. This allows the nucleophilic moiety to react fully as the free Lewis base.

In a particular instance, the nucleophilic compound is an amino acid. Any of the naturally-occurring or non-natural amino acids may be used, but an amino acid selected from histidine, lysine, or arginine is preferred.

The pharmaceutical composition—in one preferred aspect—comprises two or more different amino acids, such as three or more different amino acids. For instance, a combination of three amino acids histidine, lysine, and arginine has been shown to be particularly effective (see example 2).

The nucleophilic compound may in some instances be a dipeptide, a tripeptide or a tetrapeptide, and is preferably a dipeptide. Dipeptides, tripeptides and tetrapeptides have the advantage that they are stronger nucleophiles than single amino acids due to the lower pKa value of the terminal amino group (Stark 1965).

Particular dipeptides of interest are selected from: Gly-Gly, Lys-Pro, Val-Pro, Ile-Pro, Tyr-Pro, Ser-Pro, Pro-Ser, Ala-Gln, Ala-Glu, Tyr-Ala, Val-Tyr, Gly-Sar, and Gly-His.

Particular tetrapeptides of interest are selected from Gly-Gly-Gly-Gly, and D-Phe-D-Phe-D-Leu-D-Lys-4-amino-piperidine-4-carboxylic acid TFA salt (Difelikefalin).

In certain instances, the nucleophilic compound is not an amino acid.

In one particular aspect, the nucleophilic compound may be selected from the group consisting of acetylcysteine (thiol nucleophile), phenelzine (hydrazine nucleophile), sitagliptin (small molecule amino group nucleophile).

In one aspect, the nucleophilic compound is not a compound of formula (I) or (II)

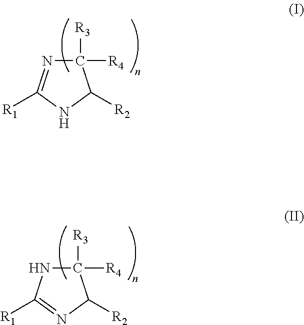

where R₁ is H or C₁-C₄ alkyl, R₂ is H, —COOH, —COO(C₁-C₄ alkyl) or —CONHR₅, where R₅ is H, C₁-C₄ alkyl, an amino acid radical, dipeptide radical or tripeptide radical, R₃ and R₄ are in each case independently of one another H or OH, n is 1, 2 or 3, or a pharmaceutically acceptable salt, stereoisomer or solvate thereof. As shown in Example 3, ectoine results in a high degree of carbamylation (%) relative to HBSS control, and is not considered to be a “nucleophilic compound” within the meaning of the present invention.

In one aspect, the nucleophilic compound is a nucleophilic small molecule. The term “small molecule” is defined by organic molecules having a MW of less than 500 g/mol.

The nucleophilic small molecule may comprise a primary amine nucleophilic moiety (i.e. —NH₂). In this case, the nucleophilic small molecule may be selected from aspartame, anthranilic acid, N-β—aminoethyl-Gly, sitagliptin, saxagliptin, linagliptin, gemigliptin, alogliptin, rimantandine, trelagliptin, omarigliptin, evogliptin, amlodipine, methyldopa, bestatin, gentamycin, cycloserine, or ampicillin.

The nucleophilic small molecule may—alternatively or additionally—comprise a secondary amine (>NH) nucleophilic moiety, preferably a pyrrolidinyl, piperidinyl or piperazinyl moiety In this case, the nucleophilic small molecule may be selected from tenegliptin, gosogliptin, ephedrine, flurosemide, salbutamol, ketamine or ciprofloxacin.

The nucleophilic small molecule may—alternatively or additionally—comprise a guanidino (—NH—C(═NH)—NH₂) nucleophilic moiety. In this case, the nucleophilic small molecule may be selected from metformin, buformin, phenformin, proguanil, chlorproguanil or chlorhexidine.

The nucleophilic small molecule may—alternatively or additionally—comprise an amidino (—CH2-C(═NH)—NH₂) nucleophilic moiety, and may preferably be selected from pentamidine, diminazene, imidocarb or xylamidine.

The nucleophilic small molecule may—alternatively or additionally—comprise a thiol (—SH) nucleophilic moiety, and may preferably be selected from acetylcysteine or captopril.

The nucleophilic small molecule may—alternatively or additionally—comprise a hydrazino (—NH—NH₂) nucleophilic moiety. In this case, the nucleophilic small molecule may be selected from phenelzine, hydralazine, dihydralazine or endralazine.

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

Typically, the nucleophilic compound is present in the pharmaceutical composition in a concentration of 0.1-10% w/w, preferably 0.5-4% w/w, more preferably 1-3% w/w.

The pharmaceutical compositions according to the invention are preferably intended for topical application. They may be in any form such as solutions, suspensions, emulsions, pastes, ointments, gels, creams, lotions, powders, soaps, surfactant-containing cleansing preparations, topical patches, oils, foams and sprays. Preferably, the pharmaceutical composition is in the form of a topical cream or lotion, in particular an oil-in-water cream. The pharmaceutical compositions according to the invention may also be given by other routes of administration, including but not limited to, subcutaneous injections.

The pharmaceutical composition may be applied to the skin with a concentration of the nucleophile ranging between 0.001 mg/cm² and 5 mg/cm² skin surface, preferably between 0.003 mg/cm² and 1 mg/cm² skin surface, and more preferably 0.005 mg/cm² and 0.5 mg/cm² skin surface.

The pharmaceutical compositions of the present invention may comprise components other than the nucleophilic compound described herein, i.e., one or more excipients. Excipients may be carriers, adjuvant and/or vehicles. Suitable excipients can be sterile liquids, such as water and oils, including those of petroleum, animal, vegetable or synthetic origin, such as peanut oil, soybean oil, mineral oil, sesame oil and the like, disgregants, wetting agents or diluents. The selection of these excipients and the amounts to be used will depend on the form of application of the pharmaceutical composition.

In one preferred aspect, the pharmaceutical composition further comprises one or more anti-inflammatory agents, such as a corticosteroid, a calcineurin inhibitor, a PDE4 inhibitor or a Janus Kinase Inhibitor, preferably a corticosteroid selected from clobetasol, mometasone, betamethasone or hydrocortisone.

A particular pharmaceutical composition according to the invention is a topical cream comprising (in % w/w):
Histidine 0.1-1%
Arginine 0.5-1.5%
Lysine 0.50-1.5%
optionally, corticosteroid 0.01-1.0%

by weight of the entire pharmaceutical composition.

Method of Treatment

A method is provided for treating a condition, in particular a dermatological condition, involving cutaneous or connective tissue damage in a patient. The method comprising administering a pharmaceutical composition comprising a nucleophilic compound capable of inhibiting carbamylation to a patient in need of such a treatment. The “nucleophilic compound” used in this method is as set out above.

As used herein, the terms “treat”, “treating” and “treatment” include in general the eradication, removal, reversion, alleviation, modification, or control of a condition after its onset. The term “treatment” includes preventative treatment (i.e. prior to onset of a condition). The terms “treat”, “treating” and “treatment” may specifically refer to the treatment or reduction of damage to cutaneous or connective tissue. Also, the terms “treat”, “treating” and “treatment” may refer to the treatment or reduction of symptoms, in particular those associated with cutaneous or connective tissue damage.

In a particular embodiment, “treat”, “treating” and “treatment” include reduction of changes/damage to elastin, collagen, filaggrin and extracellular matrix protein 1 (all of which are proteins important for tissue integrity).

The term “pharmaceutical composition” used herein include any composition manufactured for any use, other than as food, wherein a nucleophile according to the invention is used on or in the body to prevent, diagnose, alleviate, treat, relieve symptoms of, or cure a disease in humans or animals.

All details set out above regarding the pharmaceutical composition are also relevant for the method of treatment described herein. In particular, the pharmaceutical composition used in the method of the invention is a topical composition.

The pharmaceutical composition and the method of the invention may be used for dermatological conditions selected from the group consisting of cutaneous Lupus erythematosus, localized scleroderma, lichen planus, Dupuytren's contracture, Carpal tunnel syndrome, morphea, acquired perforating dermatosis, vulvovaginal atrophy, genital psoriasis, genital atopic dermatitis and lichen sclerosus. Suitably, the lichen sclerosus is genital lichen sclerosus, preferably vulvar lichen sclerosus.

Lichen Sclerosus is known to lead to cancers, in certain cases (Paulis et. al. 2019). Oxidative stress is implicated in the pathogenesis of lichen sclerosus and potential malignancies (Sander et. al. 2004; Paulis et. al. 2019). Cyanate, which is in equilibrium with isocyanate, is known to a person skilled in the art to increase oxygen stress in cells and has been shown to be carcinogenic. In one embodiment, therefore, the pharmaceutical composition for use or the method disclosed herein is for the treatment of cancer, in particular cancer caused by lichen sclerosus, such as e.g. squamous cell carcinoma, by scavenging cyanate.

In another embodiment, the pharmaceutical composition for use or the method disclosed herein is for the treatment of psoriasis and atopic dermatitis in the genital region, which presents with cutaneous and connective tissue damage, and are particularly difficult to treat in patients with incontinence. In both diseases it is known that the formation of filaggrin in the epidermal stratum granulosum is significantly decreased (Thyssen and Maibach 2014). Degradation of filaggrin from carbamylation may be a causal factor, and treatment with an isocyanate scavenger according to the invention, may thus treat the cutaneous and connective tissue damage associated with these diseases.

Treatment with the pharmaceutical composition should take place so as to provide an “effective” amount or a “therapeutically effective amount” of the nucleophilic compound to the patient (i.e. a nontoxic but sufficient amount of the drug or agent to provide the desired effect). In the therapy of the present invention, an “effective amount” of a nucleophilic compound or a derivative thereof is the amount of that compound that is effective to provide the desired effect. The amount that is “effective” will vary from subject to subject, depending on the age and general condition of the individual, the particular active agent or agents, and the like. Thus, it is not always possible to specify an exact “effective amount”. However, an appropriate “effective” amount in any individual case may be determined by one of ordinary skill in the art using routine experimentation. For instance, administration may be at regular intervals (e.g. once-daily, twice-daily etc.) or in connection with a particular event (e.g. post-urination), or a combination of such administrations.

In a particularly interesting method, the treatment comprises administering said nucleophilic compound simultaneously or consecutively with an anti-inflammatory agent, such as a corticosteroid selected from clobetasol, mometasone, betamethasone or hydrocortisone, suitably (but not necessarily) in the same pharmaceutical composition.

The cutaneous or connective tissue to be treated with the pharmaceutical composition of the invention may be fibrous connective tissue, preferably comprising collagen and/or elastin. Suitably, the treatment prevents degradation of anchoring filaments. The tissue to be treated also comprises various other components known to a person skilled in the art, including but not limited to, blood vessels and nerve fibers, which may also be the target of the treatment.

### EXAMPLES

### Example 1: Topical Cream

An oil-in-water cream base, well-known to a person skilled in the art, containing L-Histidine (0.50% w/w), L-Arginine (0.75% w/w) and L-Lysine hydrochloride (0.70%).

### Example 2: Clinical Case of Lichen Sclerosus

Case Presentation

The patient is 74-year-old woman diagnosed with lichen sclerosus for 6 years. Biopsies have confirmed the diagnosis. The patient has a tendency for stress incontinence.

The disease has had a steady progressing course, and during the 6 years has never been in complete remission. The symptoms have been dominated by pain and to a lesser degree itching. The initial objective symptoms were white and red plaques in the vulva region followed by erosions, fissures and ulcerations. Increasing severe subjective symptoms in the form of pain during e.g. urination, walking or bicycle riding. The skin changes have severely affected quality of life and sexual function. Anatomical changes are observed, including sclerosis of the labia minor, burying of the clitoris, and synechia in inflamed areas around the vagina.

Clinical History

The patient has been under medical supervision since the disease started. She has carefully kept a diary including regular photos and registration of pain on a Visual Analog Scale (VAS) with a score from 0-10. Initially, the VAS score was 7 before initiation of treatment with the amino acid cream of the present invention.

Pharmacological Treatments:

Six months prior to starting treatment with the amino acid cream of the invention she restarted maintenance treatment with clobetasol propionate ointment twice daily for 12 weeks without satisfactory effect. Treatment was continued once daily, three times weekly during the course of the therapeutic intervention with the amino acid cream.

Therapeutic Intervention

The patient started treatment with a cream containing a total of 1.95% of nucleophilic amino acids (0.5% histidine, 0.7% lysine, 0.75% arginine). The cream was applied topically to the vulvar region twice daily, supplemented with application after each urination. A total of approximately 60 g of the amino acid cream has been used monthly.

Results

Treatment with the amino acid cream showed rapid onset of effect on pain during urination. After 1 week of treatment there was a significant decrease in pain. After 3 weeks treatment the patient experienced complete remission of symptoms with a VAS score of 0-1, allowing normal daily routines without pain. Objective observations after 6 weeks of treatment showed a marked improvement. The skin was in general pale and smooth, and all fissures and ulcerations were healed without scarring, and synechia was minimized. The inflamed red area at the vaginal introitus persisted. After a total of 3 months of treatment the patient is in complete remission and without pain symptoms for the first time in 6 years. No inflammation or redness is present. The described sclerotic areas are unchanged. Physical capabilities have normalized and sensation in the treatment area has returned.

### Example 3: In Vitro Protein Carbamylation Assay

200 μL of a 0.5% BSA solution, 200 μL of test solution and 100 μL of a 5 μCi/mL ¹⁴C-Potassium Cyanate solution was mixed in an Eppendorf tube (corresponding to a final concentration of 75 μM BSA, 25 μM ¹⁴C-Potassium Cyanate, and 10 mM nucleophile). Samples run in triplicates (n=3). The Eppendorf tubes were incubated at 37° C. for 72 hours (if not otherwise stated).

After incubation a volume of 100 μL of each sample was transferred to a new Eppendorf tube containing 100 μL of a 10% trichloroacetic acid (TCA) and kept at 4° C. overnight (for precipitation of BSA). Subsequently, the precipitated BSA was separated from the buffer by centrifugation (10000 rpm, 30 seconds). The supernatant was removed and the BSA pellet was resuspended in 200 μL of a 10% solution of cold TCA (4° C.). The pellet was recovered by centrifugation (10000 rpm, 30 seconds) and supernatant was discarded. The BSA pellet was resuspended in 100 μL purified water (MilliQ) and transferred to a scintillation vial. The Eppendorf tube (previously containing the BSA pellet) was rinsed with an additional 100 μL purified water which al was transferred to the relevant scintillation vial. For scintillation counting, 2 mL scintillation fluid (UltimaGold, PerkinElmer) was added to each scintillation vial and scintillation counting was done on a TRI-Carb 2910 Scintillation counter (PerkinElmer). Results for different nucleophiles are shown in table 1

TABLE 1
In vitro protein carbamylation assay
results for different nucleophiles

| | Degree of carbamylation | |
|---|---|---|
| | (% relative to HBSS | |
| Nucleophile | control) | SD |
| HBSS control | 100 | 5.45 |
| N-β-aminoethyl-Gly | 29.7 | 0.34 |
| Gly-Gly | 26.6 | 0.11 |
| Lys-Pro | 28.6 | 0.75 |
| Val-Pro | 22.1 | 0.62 |
| Ile-Pro | 41.4 | 1.15 |
| Tyr-Pro | 32.8 | 0.88 |
| Ser-Pro | 26.4 | 0.57 |
| Pro-Ser | 41.9 | 1.15 |
| Ala-Gln | 34.8 | 0.54 |
| Ala-Glu | 20.4 | 0.20 |
| Tyr-Ala | 26.1 | 0.25 |
| Val-Tyr | 28.9 | 0.30 |
| Gly-Ser | 33.1 | 0.20 |
| Gly-His | 22.6 | 0.20 |
| Bestatin | 53.4 | 1.93 |
| Ectoine | 97.9 | 0.50 |
| Metformin*HCl^{a.b} | 71.4 | 0.87 |
| Histidine | 61.1 | 0.54 |
| Arg | 52.4 | 1.45 |
| Arg*HCl | 66.1 | 3.89 |
| Phenelzine sulfate^{b} | 9.8 | 0.28 |
| Acetylcysteine | 14.3 | 0.11 |
| Rimantadine*HCl^{a.b} | 78.5 | 0.84 |
| Aspartame | 30.4 | 0.91 |
| Pentamidine diisethionate^{b} | 43.1 | 2.67 |
| Sitagliptine phosphate^{b} | 48.6 | 0.45 |
| Difelikefalin (D-Phe-D-Phe-D- | 23.2 | 1.1 |
| Leu-D-Lys-4-amino- | | |
| piperidine-4-carboxylic acid | | |
| TFA salt)^{b} | | |
| Tetraglycine | 24.9 | 0.45 |

^{a}Dissolved in HBSS:DMSO 90:10;
^{b}Neutralized by addition of equimolar amounts of NaOH before use.

As evident from table 1, a wide range of different compounds with various functional groups can act as nucleophiles according to the invention.

### Example 4: In Vitro Protein Carbamylation Assay Using Reconstructed Human Skin

Procedure for Preparation and Treatment of Reconstructed Human Skin Cultures

Epiderm Full Thickness (Epiderm FT, MatTek Corp., USA) tissues were transferred to B6 trays containing EpiDerm™ Full Thickness Maintenance Medium (MatTek Corp., USA) immediately upon arrival and incubated for 24 hours at 37° C. and 5% CO₂. After the initial 24-hour incubation the carbamylation experiment was started by replacing the maintenance medium with 5 mL fresh medium on the basolateral side and adding 400 μL of the different treatment solutions to the apical side (see list of treatment solutions below) and incubated at 37° C. and 5% CO₂. The carbamylation experiment was continued for six days with replacement of maintenance medium on the basolateral side and treatment solutions on the apical side every other day (day=0, 2 and 4). At day 6, the treatment solutions and the maintenance medium were removed from the tissues, and pre-warmed phosphate buffered saline (DPBS, Sigma-Aldrich) was added and removed twice before the tissues were submitted for ELISA and histological analysis. The 7 different conditions were tested on four different tissues where one tissue from each treatment was submitted for ELISA and the remaining three tissues were submitted for histological analysis.

Overview of Treatment Solutions Added to the Apical Compartment
1. Control (no treatment)
2. 400 μL of 0.5 M urea:isocyanate 99:1
3. 400 μL of 0.5 M urea:isocyanate 99:1+200 μL 2% Ala-Gln solution
4. 400 μL of 0.5 M urea:isocyanate 99:1+200 μL 2% acetylcystein solution
400 μL of 0.5 M urea:isocyanate 99:1+200 μL 2% phenelzine sulfate solution (neutralized with 1 equivalent of NaOH before use)
6. 400 μL of 0.5 M urea:isocyanate 99:1+200 μL 2% His solution
7. 400 μL of 0.5 M urea:isocyanate 99:1+200 μL 2% Sitagliptin solution

A 99:1 mixture of urea and isocyanate was used for the experiment in order to mimic the equilibrium between the two species under biological conditions. The concentration used was selected to resemble the approximate concentration of urea in urine (Liu et. al. 2012).

Protein Extraction Procedure for Protein Carbamylation ELISA

Experimental procedure for protein extraction was modified from Ross-Hansen et al., 2014 and Palosuo et al., 1998. The cell covered EpidermFT filters (Mattek) were cut from the plastic insertion ring, and the cell layer gently peeled of the membrane with a scalpel. The cell layer was placed in a mortar, added 250 μl extraction buffer (1 M potassium phosphate, 2 mM Na₂EDTA, 0.1% sodium azide, 1 Complete Mini Protease inhibitor tablet pr 50 ml, pH 6,9) plus 0,07 g of quartz sand (Merck cat no 1.075360250) and ground until a smooth paste was obtained. The paste was transferred to 1.5 ml tubes and the mortars washed with 250 μl extra buffer. The samples were chilled and sonicated at max settings for 5 min. The samples were incubated 1 hour, RT, rotating. Subsequently samples were rechilled and resonicated. The mixture was centrifuged 5000×g for 15 min and the supernatant was decanted into fresh tubes. For delipidation, 400 μl of 2-bromo-chloro-1,1,1-trifluoroethane (Merck cat no B4388-125) was added and the tubes were rotated on a mixer for 1 hour. After centrifugation at 5000×g for 15 min, the supernatant was collected, and the interphase and lipid solvent phase was discarded. The protein extracts were dialysed overnight in PBS in Slide-A-Lyzer 7K casettes (Thermo Fisher cat no 66370), and stored at −20° C. Samples were analysed undiluted in a Protein Carbamylation Sandwich ELISA (Cell Biolabs Inc cat no STA-877). Results are shown in FIG. 2.

As evident from FIG. 2, a range of chemically different nucleophiles with various functional groups can, according to the invention, act as scavengers of isocyanate in a reconstructed human skin model. For the control samples, treatment with a 99:1 mixture of urea and isocyanate did result in significant carbamylation as expected compared with control samples without urea:isocyanate treatment.

Procedure for Histological Analysis of Skin Samples

EpiDermFT membranes were app. 1 cm in diameter and were fixed in formalin for 5 days. Membranes were cut into halves using a scissors and placed into histogel matrix aiming at having the cut surface as the tissue sectioning surface. The samples were dehydrated in alcohol and xylene and embedded in paraffin using a standard paraffin embedding procedure. Sections were cut at 4 μm for Hematoxylin and Eosin (H&E) staining and immunohistochemistry (IHC). Images were acquired using a 20× objective with a Zeiss AxioScan. Representative areas were selected for presentation.

Generally, the EpiDermFT skin samples contain three characteristic compartments: epidermis with cornified layers, a thick layer of dermis, and a lower dermis layer with increased cell density.

As evident from FIG. 3, treatment with the above mentioned nucleophiles also results in reduction of changes and damage to the skin due to urea:isocyanate. The control sample without any treatment show intact skin with the epidermis attached to the dermis. The control sample treated with urea:isocyanate did not show any attached epidermis, indicating that the treatment with urea:isocyanate causes severe cutaneous and connective tissue damage, conceivably due to carbamylation of the anchoring fibers, which bind the dermis and epidermis together. In all groups also treated with different nucleophiles, attachment of epidermis was improved to varying degree compared to the control sample treated with only urea:isocyanate. These results show that treatment with the different nucleophiles can counter the cutaneous and connective tissue damage caused by carbamylation. Treatment with Sitagliptin showed the least effect, which fits with the results from the ELISA (FIG. 2), where Sitagliptin did not show complete inhibition of protein carbamylation like the other nucleophiles did.

The Following Numbered Aspects are Provided:
Aspect 1. A pharmaceutical composition comprising a nucleophilic compound capable of inhibiting carbamylation, for use in the treatment of a condition involving cutaneous or connective tissue damage.
Aspect 2. A method for treating a dermatological condition involving cutaneous or connective tissue damage in a patient, said method comprising administering a pharmaceutical composition comprising a nucleophilic compound capable of inhibiting carbamylation to a patient in need of such a treatment.
Aspect 3. The pharmaceutical composition for use according to aspect 1 or the method according to aspect 2, wherein said composition is a topical composition.
Aspect 4. The pharmaceutical composition for use or the method according to any one of the preceding aspects, wherein the condition is a dermatological condition.
Aspect 5. The pharmaceutical composition for use or the method according to any of the preceding aspects, wherein the composition is applied to the skin with a concentration of the nucleophile ranging between 0.001 mg/cm² and 5 mg/cm² skin surface, preferably between 0.003 mg/cm² and 1 mg/cm² skin surface, and more preferably 0.005 mg/cm² and 0.5 mg/cm² skin surface.
Aspect 6. The pharmaceutical composition for use or the method according to any one of the preceding aspects, wherein the dermatological condition is selected from the group consisting of cutaneous Lupus erythematosus, localized scleroderma, lichen planus, Dupuytren's contracture, Carpal tunnel syndrome, morphea, acquired perforating dermatosis, vulvovaginal atrophy, genital psoriasis, genital atopic dermatitis and lichen sclerosus.
Aspect 7. The pharmaceutical composition for use or the method according to any one of the preceding aspects, wherein said nucleophilic compound results in a degree of BSA carbamylation of less than 80%, preferably less than 70%, more preferably less than 60%, even more preferably less than 50% as measured by the in vitro protein carbamylation assay provided herein.
Aspect 8. The pharmaceutical composition for use or the method according to any one of the preceding aspects, wherein the nucleophilic compound is an organic molecule comprising at least one nucleophilic moiety.
Aspect 9. The pharmaceutical composition for use or the method according to aspect 6, wherein said at least one nucleophilic moiety is selected from primary amine (—NH₂), secondary amine (—NHR₁—), guanidino (—NR₁C(NR₂)NR₃R₄), amidino (—C(NR₂)NR₃R₄), hydrazino (R₁—NR₂—NR₃R₄) or thiol (—SH).
Aspect 10. The pharmaceutical composition for use or the method according to any one of aspects 6-7, wherein said at least one nucleophilic moiety is in its unprotonated form, in said composition or said method.
Aspect 11. The pharmaceutical composition for use or the method according to any one of the preceding aspects, wherein the nucleophilic compound is an amino acid such as an amino acid selected from histidine, lysine, or arginine.
Aspect 12. The pharmaceutical composition for use or the method according to any one of the preceding aspects, wherein the pharmaceutical composition comprises two or more different amino acids, such as three or more different amino acids.
Aspect 13. The pharmaceutical composition for use or the method according to aspect 10, wherein the pharmaceutical composition comprises histidine, lysine, and arginine, or a combination of one or more of histidine, lysine, and arginine.
Aspect 14. The pharmaceutical composition for use or the method according to any one of the preceding aspects, wherein the nucleophilic compound is a dipeptide, a tripeptide or a tetrapeptide, preferably a dipeptide.
Aspect 15. The pharmaceutical composition for use or the method according to any one of the preceding aspects, wherein the nucleophilic compound is a dipeptide or tetrapeptide selected from: Gly-Gly, Lys-Pro, Val-Pro, Ile-Pro, Tyr-Pro, Ser-Pro, Pro-Ser, Ala-Gln, Ala-Glu, Tyr-Ala, Val-Tyr, Gly-Sar, Gly-His, Gly-Gly-Gly-Gly, D-Phe-D-Phe-D-Leu-D-Lys-4-amino-piperidine-4-carboxylic acid TFA salt.
Aspect 16. The pharmaceutical composition for use or the method according to any one of the preceding aspects, wherein the nucleophilic compound is not a compound of formula (I) or (II):

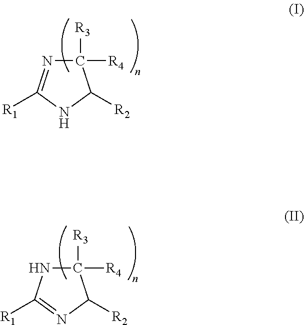

in which R₁ is H or C₁-C₄ alkyl,
R₂ is H, —COOH, —COO(C₁-C₄ alkyl) or —CONHR₅, where R₅ is H, C₁-C₄ alkyl, an amino acid radical, dipeptide radical or tripeptide radical, R₃ and R₄ are in each case independently of one another H or OH, n is 1, 2 or 3, or a pharmaceutically acceptable salt, stereoisomer or solvate thereof.
Aspect 17. The pharmaceutical composition for use or the method according to any one of the preceding aspects, wherein the nucleophilic compound is a nucleophilic small molecule.
Aspect 18. The pharmaceutical composition for use or the method according to any one of the preceding aspects, wherein the nucleophilic small molecule comprises a primary amine nucleophilic moiety.
Aspect 19. The pharmaceutical composition for use or the method according to aspect 18, wherein the nucleophilic small molecule is selected from aspartame, anthranilic acid, N-β-aminoethyl-Gly, sitagliptin, saxagliptin, linagliptin, gemigliptin, alogliptin, rimantandine, trelagliptin, omarigliptin, evogliptin, amlodipine, methyldopa, bestatin, gentamycin, cycloserine, gabapentin, pregabalin, or ampicillin.
Aspect 20. The pharmaceutical composition for use or the method according to any one of aspects 1-17, wherein the nucleophilic small molecule comprises a secondary amine nucleophilic moiety, preferably a pyrrolidinyl, piperidinyl or piperazinyl moiety.
Aspect 21. The pharmaceutical composition for use or the method according to aspect 20, wherein the nucleophilic small molecule is selected from tenegliptin, gosogliptin, ephedrine, flurosemide, salbutamol, ketamine or ciprofloxacin.
Aspect 22. The pharmaceutical composition for use or the method according to any one of aspects 1-17, wherein the nucleophilic small molecule comprises a guanidino nucleophilic moiety.
Aspect 23. The pharmaceutical composition for use or the method according to aspect 22, wherein the nucleophilic small molecule is selected from metformin, buformin, phenformin, proguanil, chlorproguanil or chlorhexidine.
Aspect 24. The pharmaceutical composition for use or the method according to any one of aspects 1-17, wherein the nucleophilic small molecule comprises an amidino nucleophilic moiety, preferably wherein the nucleophilic small molecule is selected from pentamidine, diminazene, imidocarb or xylamidine.
Aspect 25. The pharmaceutical composition for use or the method according to any one of aspects 1-17, wherein the nucleophilic small molecule comprises a thiol nucleophilic moiety, preferably wherein the nucleophilic small molecule is selected from acetylcysteine or captopril.
Aspect 26. The pharmaceutical composition for use or the method according to any one of aspects 1-17, wherein the nucleophilic small molecule comprises a hydrazino nucleophilic moiety.
Aspect 27. The pharmaceutical composition for use or the method according to aspect 26, wherein the nucleophilic small molecule is selected from phenelzine, hydralazine, dihydralazine or endralazine.
Aspect 28. The pharmaceutical composition for use or the method according to any one of the preceding aspects, wherein the lichen sclerosus is genital lichen sclerosus, preferably vulvar lichen sclerosus.
Aspect 29. The pharmaceutical composition for use or the method according to any one of the preceding aspects, wherein said composition further comprises an anti-inflammatory agent, such as a corticosteroid, a calcineurin inhibitor, a PDE4 inhibitor, or a Janus Kinase Inhibitor, preferably a corticosteroid selected from clobetasol, mometasone, betamethasone or hydrocortisone.
Aspect 30. The pharmaceutical composition for use or the method according to any one of the preceding aspects, wherein said treatment comprises administering said nucleophilic compound simultaneously or consecutively with an anti-inflammatory agent, such as a corticosteroid selected from clobetasol, mometasone, betamethasone or hydrocortisone.
Aspect 31. The pharmaceutical composition for use or the method according to any one of the preceding aspects, being in the form of a topical cream, gel or lotion, in particular an oil-in-water cream.
Aspect 32. The pharmaceutical composition for use or the method according to any one of the preceding aspects, wherein the nucleophilic compound is present in the composition in a concentration of 0.1-10% w/w, preferably 0.5-4% w/w, more preferably 1-3% w/w.
Aspect 33. The pharmaceutical composition for use or the method according to any one of the preceding aspects, being a topical cream comprising (in % w/w):
Histidine 0.1-1%
Arginine 0.5-1.5%
Lysine 0.50-1.5%
optionally, corticosteroid 0.01-1.0%
Aspect 34. A pharmaceutical composition comprising a nucleophilic compound capable of inhibiting carbamylation and an anti-inflammatory agent, such as a corticosteroid.
Aspect 35. The pharmaceutical composition according to aspect 33, wherein the nucleophilic compound is as defined in any one of aspects 1-27.

### REFERENCES

Bunker C B, Patel N, Shim T N. Urinary voiding symptomatology (micro-incontinence) in male genital lichen sclerosus. Acta Derm Venereol. 2013; 93(2):246-248.
Christmann-Schmid C, Hediger M, GrOger S, Krebs J, Günthert A R; In cooperation with the Verein Lichen sclerosus. Vulvar lichen sclerosus in women is associated with lower urinary tract symptoms. Int Urogynecol J. 2018; 29(2):217-221.
Favoino E, Prete M, Vettori S, et al. Anti-carbamylated protein antibodies and skin involvement in patients with systemic sclerosis: An intriguing association. PLoS One. 2018; 13(12)
Godoy, Charles & Teodoro, Walcy & Velosa, Ana & Garippo, Ana & Eher, Esmeralda & Parra, Edwin & Sotto, Mirian & Capelozzi, Vera. (2015). Unusual remodeling of the hyalinization band in vulval lichen sclerosus by type V collagen and ECM 1 protein. Clinics (São Paulo, Brazil), 70(5), 356-62.
Jaisson, S., Lorimier, S., Ricard-Blum, S., Sockalingum, G. D., Delevallee-Forte, C., Kegelaer, G., Manfait, M., Garnotel, R., and Gillery, P. (2006), Impact of carbamylation on type I collagen conformational structure and its ability to activate human polymorphonuclear neutrophils. Chem. Biol. 13, 149-159.
Jaisson, S., Larreta-Garde, V., Bellon, G., Hornebeck, W., Garnotel, R., and Gillery, P. (2007). Carbamylation differentially alters type I collagen sensitivity to various collagenases. Matrix Biol. 26, 190-196.
Jaisson, S., Sartelet, H., Perreau, C., Blanchevoye, C., Garnotel, R., and Gillery, P. (2008). Involvement of lysine 1047 in type I collagen-mediated activation of polymorphonuclear neutrophils. FEBS J. 275, 3226-3235.
Liu L, Mo H, Wei S, Raftery D. Quantitative analysis of urea in human urine and serum by 1H nuclear magnetic resonance. Analyst. 2012; 137(3):595-600.
Pietrement, C., Gorisse, L., Jaisson, S., Gillery, P. Chronic increase of urea leads to carbamylated proteins accumulation in tissues in a mouse model of CKD, PLoS One 8 (2013).
Paulis, G., Berardesca, E. Lichen sclerosus: the role of oxidative stress in the pathogenesis of the disease and its possible transformation into carcinoma. Research and Reports in Urology 2019:11 223-232.
Sander, C. S., Ali, I., Dean, D., Thiele, J. J., Woinarowska, F., Oxidative stress is implicated in the pathogenesis of lichen sclerosus. British J. Dermatol., Volume 151, Issue 3, September 2004, pages 627-635.
Shi J, van Veelen P A, Mahler M, et al. Carbamylation and antibodies against carbamylated proteins in autoimmunity and other pathologies. Autoimmun Rev. 2014; 13:225-30.
Stark, G. R., Reactions of Cyanate with Functional Groups of Proteins. III. Reactions with Amino and Carboxyl Groups, Biochemistry 1965 4 (6), 1030-1036.
Taga, Y., Hydroxyhomocitrulline Is a Collagen-Specific Carbamylation Mark that Affects Cross-link Formation, Cell Chemical Biology 24, 1276-1284, 2017.
Thyssen, J. P, Maibach H. I., Filaggrin; Basic Science, Epidemiology, Clinical Aspects and Management, Springer 2014.
Tran D A, Tan X, Macri C J, Goldstein A T, Fu S W. Lichen Sclerosus: An autoimmunopathogenic and genomic enigma with emerging genetic and immune targets. Int J Biol Sci 2019; 15(7):1429-1439.
Wang, Z., Nicholls, S., Rodriguez, E. et al. Protein carbamylation links inflammation, smoking, uremia and atherogenesis. Nat Med 13, 1176-1184 (2007).
Ziegelasch M, van Delft M A, Wallin P, et al. Antibodies against carbamylated proteins and cyclic citrullinated peptides in systemic lupus erythematosus: results from two well-defined European cohorts. Arthritis Res Ther. 2016; 18(1): 289.

The invention has been described with reference to a number of examples and embodiments. However, the true scope of the invention is set out in the enclosed independent claims.

1. A pharmaceutical composition comprising a nucleophilic compound capable of inhibiting carbamylation, for use in the treatment of lichen sclerosus.

2. A method for treating lichen sclerosus in a patient, said method comprising administering a pharmaceutical composition comprising a nucleophilic compound capable of inhibiting carbamylation to a patient in need of such a treatment.

3. The pharmaceutical composition for use according to claim 1, wherein said composition is a topical composition.

4. The pharmaceutical composition for use or the method according to claim 1, wherein said nucleophilic compound results in a degree of BSA carbamylation of less than 80%, preferably less than 70%, more preferably less than 60%, even more preferably less than 50% as measured by the in vitro protein carbamylation assay provided herein.

5. The pharmaceutical composition for use or the method according to claim 1, wherein the nucleophilic compound is a dipeptide, a tripeptide or a tetrapeptide, preferably a dipeptide.

6. The pharmaceutical composition for use or the method according to claim 1, wherein the nucleophilic compound is a dipeptide or tetrapeptide selected from: Gly-Gly, Lys-Pro, Val-Pro, Ile-Pro, Tyr-Pro, Ser-Pro, Pro-Ser, Ala-Gln, Ala-Glu, Tyr-Ala, Val-Tyr, Gly-Sar, Gly-His, Gly-Gly-Gly-Gly, D-Phe-D-Phe-D-Leu-D-Lys-4-amino-piperidine-4-carboxylic acid TFA salt, or wherein the nucleophilic compound is selected from the group consisting of acetylcysteine, phenelzine and sitagliptin.

7. The pharmaceutical composition for use or the method according to claim 1, wherein the nucleophilic compound is not a compound of formula (I) or (II): 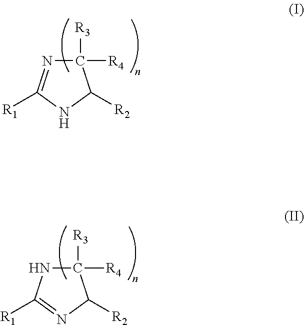
in which R₁ is H or C₁-C₄ alkyl,
R₂ is H, —COOH, —COO(C₁-C₄ alkyl) or —CONHR₅, where R₅ is H, C₁-C₄ alkyl, an amino acid radical, dipeptide radical or tripeptide radical, R₃ and R₄ are in each case independently of one another H or OH, n is 1, 2 or 3, or a pharmaceutically acceptable salt, stereoisomer or solvate thereof.

8. The pharmaceutical composition for use or the method according to claim 1, wherein the lichen sclerosus is genital lichen sclerosus, preferably vulvar lichen sclerosus.

9. The pharmaceutical composition for use or the method according to claim 1, wherein the nucleophilic compound is selected from the group of acetylcysteine, phenelzine, sitagliptin, or Ala-Gln and wherein said composition is a topical composition.

10. The pharmaceutical composition for use or the method according to claim 1, wherein the invention is a topical cream comprising (in w/w %):
Histidine 0.1-1%
Arginine 0.5-1.5%
Lysine 0.50-1.5%
optionally, corticosteroid 0.01-1.0%
by weight of the entire pharmaceutical composition.
